(19) United States
(12) Patent Application Publication
(10) Pub. No.: US 2021/0395178 A1
(43) Pub. Date: Dec. 23, 2021

(54) SELECTIVE DIMERIZATION AND ETHERIFICATION OF ISOBUTYLENE VIA CATALYTIC DISTILLATION

(71) Applicant: LUMMUS TECHNOLOGY LLC, Bloomfield, NJ (US)

(72) Inventors: Rosette Barias, Spring, TX (US); Romain Lemoine, Houston, TX (US)

(73) Assignee: LUMMUS TECHNOLOGY LLC, Bloomfield, NJ (US)

(21) Appl. No.: 17/290,135

(22) PCT Filed: Oct. 31, 2019
(86) PCT No.: PCT/US2019/059171

§ 371 Date: Apr. 29, 2021

Related U.S. Application Data
(60) Provisional application No. 62/753,851, filed on Oct. 31, 2018.

(51) Int. Cl. C07C 41/09; B01J 8/04; C07C 41/42; C07C 7/04; C07C 2/08; B01D 3/00; B01D 3/14

(52) U.S. Cl. CPC C07C 41/09; B01J 8/04; B01J 8/0496; C07C 41/42; C07C 7/04; B01J 2208/00628; B01D 3/009; B01D 3/143; B01J 2208/00017; B01J 2208/00539; C07C 2/08

(57) ABSTRACT

A process for the selective dimerization and etherification of isoolefins, including feeding a mixed C4 stream and an oxygenate stream to a first fixed bed reactor containing a first catalyst, producing a first reactor effluent comprising dimers of the isoolefin, unreacted C4s, and unreacted oxygenates. Feeding the first reactor effluent directly to a second fixed bed reactor containing a second catalyst, producing a second reactor effluent containing dimers of the isoolefin, unreacted C4s, and unreacted oxygenates. Feeding the second reactor effluent to a catalytic distillation reactor system containing a third catalyst. Concurrently in the catalyst distillation reactor system reacting unreacted C4s in the presence of the third catalyst to form additional dimers of the isoolefin and/or ethers, and separating the dimers of the isoolefins from unreacted oxygenates and unreacted C4s.

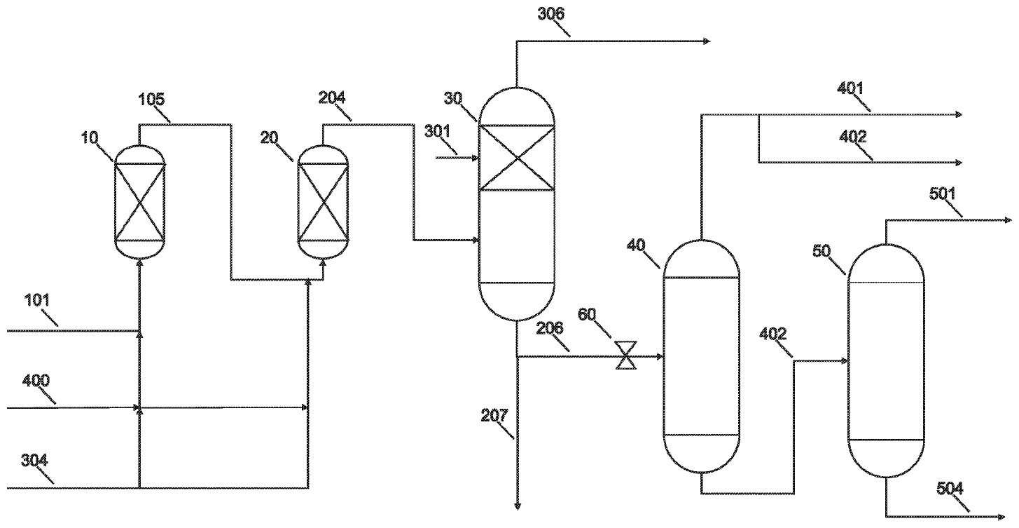

### FIELD OF THE DISCLOSURE

Embodiments disclosed herein relate generally to a process for the dimerization of isoolefins. Some embodiments herein relate to processes and apparatus for the dimerization of isoolefins, such as isobutylene, at high selectivity. Other embodiments herein relate to processes and apparatus for the concurrent dimerization and etherification of isoolefins, such as isobutylene, to form a crude ether product. Other embodiments herein relate to processes and apparatus for the selective dimerization and etherification of isobutylene via catalytic distillation.

### BACKGROUND

In order to meet the fuel blending requirements, such as octane rating or vapor pressure requirements, smaller olefin molecules may be upgraded to produce longer chain molecules. Alternatively, the smaller olefin molecules may be etherified so as to increase the oxygen content of the molecule and the resulting fuel blend.

One commonly used method of upgrading smaller olefin molecules, such as C₂ to C₅ olefins, is a dimerization reaction. Isobutylene is commercially significant in many applications. For example, isobutylene is one of the comonomers in butyl rubber. Isobutylene can also be dimerized to produce compounds that can be used as chemical feedstock for further reacting or in gasoline blending. Diisobutylene, the isobutylene dimer, is of particular commercial value in several applications. For example, diisobutylene can be used as an alkylation reaction feedstock or as an intermediate in the preparation of detergents. Diisobutylene can also be hydrogenated to pure isooctane (2,2,4-tri-methyl pentane) that is highly preferred in gasoline blending.

Dimerization reactions involve contacting an olefin with a catalyst in order to produce a longer chain molecule. A dimer can consist of two or more constituent olefin molecules. For example, dimerization is a type of dimerization reaction that is limited to a combination of only two olefin molecules. If the olefin feed contains only one type of olefin, a dimer product is formed. If the olefin feed contains two or more different olefins or olefin isomers, a codimer product may also be formed.

Specifically, C₄ olefin dimerization is widely used for producing isooctene, an intermediate that can be hydrogenated to produce isooctane, a high-value gasoline blending additive. Several representative olefin dimerization reactions are shown below:

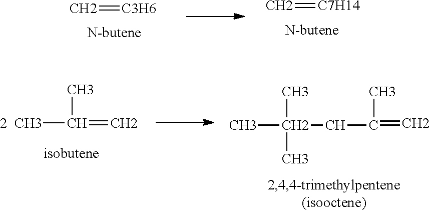

A gas phase olefin dimerization process is disclosed in U.S. Pat. Nos. 3,960,978 and 4,021,502, where C₂ to C₅ olefins, fed as either pure olefins or in admixture with paraffins, are dimerized via contacted with a zeolite fixed catalyst bed. Other dimerization processes are disclosed in, for example, U.S. Pat. Nos. 4,242,530, 4,375,576, 5,003,124, 7,145,049, 6,335,473, 6,774,275, 6,858,770, 6,936,742, 6,995,296, 7,250,542, 7,288,693, 7,319,180, 6,689,927, 6,376,731, 5,877,372, 4,331,824, 4,100,220 and U.S. Patent Application Publication Nos. 20080064911, 20080045763, 20070161843, 20060030741, 20040210093, and 20040006252, among others. Acid resin catalysts have also found use in various other petrochemical processes, including formation of ethers, hydration of olefins, esterifications, and expoxidations, such as described in U.S. Pat. Nos. 4,551,567 and 4,629,710.

Processes for dimerization of olefins over such resin catalysts require periodic shutdowns of the dimerization unit to replace and/or regenerate the catalysts. Further, such solid-catalyzed processes may require additives (“selectivators”) to promote the selectivity of the catalyst to the dimer, where the additives may result in unwanted acid throw, deactivating the catalyst, and may additionally require complicated separation processes to remove the additive from the resulting product streams.

In any type of dimerization reaction, the dimerization catalyst activity can be drastically reduced due to poisoning, fouling, and coking frequently caused by impurities present in the olefin feed stream. Furthermore, various additives and impurities that may be present in the olefin feed can participate in side reactions leading to formation of undesirable byproducts. For example, the presence of normal butene in the isobutylene dimerization process to produce isooctene dimer can lead to formation of undesirable C₈ codimers. Formation of C₈ codimers can adversely affect an operator in two major ways. First, it reduces the effective yield of the C₈ dimer target product, thus increasing the dimerization reactor feedstock and operating costs. Second, it may require additional costs associated with separation and removal of C₈ codimers from the C₈ dimer product.

Dimerization reaction additives, such as a reaction moderator, can also participate in undesirable side reactions with the olefin or with the dimerization product. Moderator is frequently added to the dimerization reaction in order to increase the dimer selectivity by limiting the extent of oligomerization reaction to the dimer stage. Suitable moderators include oxygenates, such as water, primary, secondary and tertiary alcohols and ethers. However, as a trade-off to achieving high dimer selectivity, a portion of the moderator can react with an olefin or a dimerization product to form heavy oxygenates, for example, MSBE. A representative reaction of an olefin with a moderator to form a heavy oxygenate is shown below:

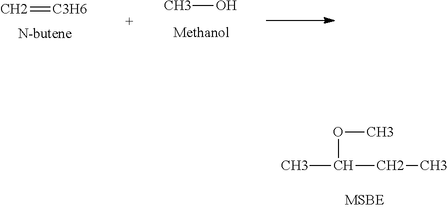

Similar to other types of side reactions, the reaction of moderator to produce heavy oxygenates, such as MSBE, can also reduce the C₈ dimer product yield and require additional separation costs in order to maintain the desired product purity.

Regarding etherification, the reaction of an alcohol and an olefin and concurrent separation of the reactants from the reaction products by fractional distillation has been practiced for some time. The process is variously described in U.S. Pat. Nos. 4,232,177; 4,307,254; 4,336,407; 4,504,687; 4,987,807; and 5,118,873.

Briefly, the alcohol and isoolefin are fed to a distillation column reactor having a distillation reaction zone containing a suitable catalyst, such as an acid cation exchange resin, in the form of catalytic distillation structure, and also having a distillation zone containing inert distillation structure. As embodied in the etherification of iC₄='s and/or iC₅='s the olefin and an excess of methanol are first fed to a fixed bed reactor wherein most of the olefin is reacted to form the corresponding ether, methyl tertiary butyl ether (MTBE) or tertiary amyl methyl ether (TAME). The fixed bed reactor is operated at a given pressure such that the reaction mixture is at the boiling point, thereby removing the exothermic heat of reaction by vaporization of the mixture. The fixed bed reactor and process are described more completely in U.S. Pat. No. 4,950,803 which is hereby incorporated by reference.

The effluent from the fixed bed reactor is then fed to the distillation column reactor wherein the remainder of the iC₄='s or iC₅='s are usually converted to the ether and the methanol is separated from the ether which is withdrawn as bottoms. The C₄ or C₅ olefin stream generally contains only about 10 to 60 percent olefin, the remainder being inerts which are removed in the overheads from the distillation column reactor.

In some cases, the distillation column reactor may be operated such that complete reaction of the isoolefin is not achieved for a particular reason and therefore there may be significant isoolefin in the overheads, that is, from 1 to 15 wt %, along with unreacted methanol.

Accordingly, there exists a continuing need for improved isoolefin dimerization catalysts, systems, and processes and isoolefin etherification catalysts, systems, and processes.

### SUMMARY OF THE DISCLOSURE

According to one or more embodiments disclosed herein is a process for the selective dimerization and etherification of isoolefins, the process comprising feeding a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates; feeding the first reactor effluent directly to a second fixed bed reactor containing a second catalyst, producing a second reactor effluent comprising dimers of the isoolefin, unreacted C4s, and unreacted oxygenates; feeding the second reactor effluent to a catalytic distillation reactor system containing a third catalyst; concurrently in the catalyst distillation reactor system; reacting unreacted C4s in the presence of the third catalyst to form additional dimers of the isoolefin and/or ethers, and separating the dimers of the isoolefins from unreacted oxygenates and unreacted C4s, producing a bottoms stream comprising the dimers of the isoolefins, any produced trimers of the isoolefins, and heavy oxygenates, and an overhead stream comprising unreacted light oxygenates and C4s.

According to one or more embodiments disclosed herein is a process for the flexible production of dimers and ethers comprising feeding a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates; feeding the first reactor effluent directly to a second fixed bed reactor containing a second catalyst, producing a second reactor effluent comprising dimers of the isoolefin, unreacted C4s, and unreacted oxygenates; feeding the second reactor effluent to a catalytic distillation reactor system containing a third catalyst; concurrently in the catalyst distillation reactor system; reacting unreacted C4s in the presence of the third catalyst to form additional dimers of the isoolefin and/or ethers, and separating the dimers of the isoolefins from uncreacted oxygenates and unreacted C4s, producing a bottoms stream comprising the dimers of the isoolefins, any produced trimers of the isoolefins, and heavy oxygenates, and an overhead stream comprising unreacted light oxygenates and C4s; and for a first period of time, feeding the oxygenate to the first and second reactor at a concentration for the oxygenate to be effective as a selectivator, producing dimers of the isoolefins; increasing the amount of oxygenates fed to the first and second reactors to a concentration for the oxygenate to be effective as a reactant, and for a second period of time producing ethers of the isoolefin.

According to one or more embodiments disclosed herein is A system for the flexible production of dimers and ethers comprising a first fixed bed reactor containing a first catalyst configured for reacting ng a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates; a second fixed bed reactor containing a second catalyst configured for reacting the first reactor effluent and producing a second reactor effluent comprising dimers of the isoolefin, unreacted C4s, and unreacted oxygenates; a catalytic distillation reactor system containing a third catalyst; concurrently in the catalyst distillation reactor system; reacting unreacted C4s in the presence of the third catalyst to form additional dimers of the isoolefin and/or ethers, and separating the dimers of the isoolefins from uncreacted oxygenates and unreacted C4s, producing a bottoms stream comprising the dimers of the isoolefins, any produced trimers of the isoolefins, and heavy oxygenates, and an overhead stream comprising unreacted light oxygenates and C4s; and for a first period of time, feeding the oxygenate to the first and second reactor at a concentration for the oxygenate to be effective as a selectivator, producing dimers of the isoolefins; increasing the amount of oxygenates fed to the first and second reactors to a concentration for the oxygenate to be effective as a reactant, and for a second period of time producing ethers of the isoolefin.

Other aspects and advantages will be apparent from the following description and the appended claims.

### BRIEF DESCRIPTION OF DRAWINGS

The FIGURE is a simplified process flow diagram of a system for dimerization and/or etherification of isoolefins according to embodiments herein.

### DETAILED DESCRIPTION

Embodiments herein relate generally to dimerization and/or etherification of isoolefins.

As used in embodiments disclosed herein, “catalytic distillation reactor system” refers to a system for concurrently reacting compounds and separating the reactants and the products using fractional distillation. In some embodiments, the catalytic distillation reactor system may comprise a conventional catalytic distillation column reactor, where the reaction and distillation are concurrently taking place at boiling point conditions. In other embodiments, the catalytic distillation reactor system may comprise a distillation column combined with at least one side reactor, where the side reactor may be operated as a liquid phase reactor or a boiling point reactor. While both catalytic distillation reactor systems described may be preferred over conventional liquid phase reaction followed by separations, a catalytic distillation column reactor may have the advantages of decreased piece count, reduced capital cost, increased catalyst productivity per pound of catalyst, efficient heat removal (heat of reaction may be absorbed into the heat of vaporization of the mixture), and a potential for shifting equilibrium. Divided wall distillation columns, where at least one section of the divided wall column contains a catalytic distillation structure, may also be used, and are considered “catalytic distillation reactor systems” herein.

The hydrocarbon feed to the reactor(s) may include purified isoolefin streams, such as a feed stream containing, isobutylene, isoamylenes, or mixtures thereof. In other embodiments, hydrocarbon feeds may include a C₄-C₅, a C₄ or a C₅ light naphtha cut. When present in mixtures, the tertiary olefins, such as isobutylene and isoamylenes, are more reactive than the normal olefin isomers and are preferentially reacted (dimerized or etherified). The isoalkanes in the C₄ to C₅ light naphtha cuts may include isobutane, isopentane or mixtures thereof, which may act as a diluent in the reactors.

In some embodiments, a C4-containing hydrocarbon stream, such as a C4 naphtha cut, a C4-C5 naphtha cut, or a C4-C6 naphtha cut may be fed to a reactor for the hydroisomerization of 1-butene to 2-butene, thus allowing for the separation of isobutylene from the linear olefin 2-butene. The hydroisomerization may be carried out in a fixed bed reactor as well as in a catalytic distillation reaction system. For example, in some embodiments, a feed containing 1-butene, 2-butene, isobutylene, n-butane, and isobutane may be fed to a catalytic distillation reaction system containing at least one bed of hydroisomerization catalyst for the concurrent hydroisomerization of 1-butene to 2-butene and the fractionation of isobutane and isobutylene, recovered as an overheads, from the heavier hydrocarbons in the feed stream, including the n-butane and 2-butene, recovered as a bottoms fraction. Feed and catalyst locations may be positioned so as to preferentially contact the 1-butene with the hydroisomerization catalyst. For example, the hydrocarbon may be fed to a location below the hydroisomerization catalyst, allowing the 1-butene to distill up into the catalyst bed while distilling the 2-butene down the column, away from the catalyst bed. In other embodiments, a hydroisomerized effluent from a fixed bed reactor may be fed to a conventional distillation column to result in similar overheads and bottoms fractions.

The resulting bottoms fraction, including the 2-butene and the n-butane, may be lean in 1-butene, isobutane, and isobutylene. For example, depending upon the severity of the distillation conditions used, the bottoms fraction may contain less than 1 weight percent total of 1-butene, isobutane, and isobutylene; less than 0.5 weight percent total in other embodiments; less than 0.1 weight percent total in other embodiments; and less than 500 ppm total in yet other embodiments.

The overheads fraction, including the isobutylene and isobutane may also contain some unreacted 1-butene. In some embodiments, the overheads fraction may contain less than 1000 ppm 1-butene; less than 500 ppm in other embodiments; less than 250 ppm in other embodiments; less than 100 ppm in other embodiments; and less than 50 ppm in yet other embodiments.

The overhead fraction may then be reacted to form desired reaction products, such as C8 hydrocarbons and C4 ethers, according to embodiments herein.

The C4 and/or C5 isoolefins may be processed according to embodiments herein to selectively dimerize the isoolefins, etherify the isoolefins, or both. Systems according to embodiments herein may be used flexibly to produce dimers during a production campaign, and when market demands change, to produce ethers during a production campaign. Catalysts used in reactors and distillation column reactors according to embodiments herein may have functionality to selectively dimerize isoolefins as well as to etherify the isoolefins. Accordingly, the process may be transitioned between dimerization and etherification readily, without the need to change catalysts. Rather, operating conditions, including temperature, pressure, residence time, and reactant concentrations, among others, may be transitioned appropriately to effect the desired reaction.

Processes disclosed herein may include any number of reactors, including catalytic distillation reactor systems, both up-flow and down-flow. Use of catalytic distillation reactor systems may prevent foulants and heavy catalyst poisons in the feed from building up within the reaction zone(s). In addition, clean reflux may continuously wash the catalytic distillation structure in the reaction zone. These factors combine to provide a long catalyst life. The heat of reaction evaporates liquid and the resulting vapor is condensed in the overhead condenser to provide additional reflux. The resulting temperature profile in the reaction zone in the catalytic distillation reaction system is much closer to an isothermal catalyst bed rather than the adiabatic temperature increase typical of conventional fixed bed reactors.

Other reactors useful in embodiments disclosed herein may include traditional fixed bed reactors, boiling point reactors, and pulsed flow reactors, where the reactant flow and product flow may be co-current or counter-current. Boiling point and pulsed flow reactors may also provide for a continuous washing of the catalyst in addition to capturing at least a portion of the heat of reaction through evaporation, allowing for an improved reactor temperature profile as compared to conventional fixed bed reactors. Reactors useful in embodiments disclosed herein may be used as a stand-alone reactor or may be used in combination with one or more reactors of the same or different type.

Any type of reactor may be used to carry out the reactions described herein. The examples of reactors suitable for carrying out the reactions involving isoolefin reactions according to embodiments herein may include distillation column reactors, divided wall distillation column reactors, traditional tubular fixed bed reactors, bubble column reactors, slurry reactors equipped with or without a distillation column, pulsed flow reactors, catalytic distillation columns wherein slurry solid catalysts flow down the column, or any combination of these reactors. Multiple reactor systems useful in embodiments disclosed herein may include a series of multiple reactors or multiple reactors in parallel for the first reaction zone. A person of ordinary skill in the art would recognize that other types of reactors may also be used.

The reactors useful in embodiments disclosed herein may include any physical devices or a combination of two or more devices, including reactors and reactor systems as described above. The reactor(s) may have various internal devices for vapor-liquid separation and vapor/liquid traffic. Reaction zones within the reactor(s) may include “wettable” structure and/or packing. Wettable structure and packing useful in embodiments disclosed herein may include various distillation structures and packing materials, which may be catalytic or non-catalytic. Suitable wettable structure and packing may include, for example, random or dumped distillation packings which are: catalytically inert dumped packings that contain higher void fraction and maintain a relatively large surface area, such as, Berl Saddles (Ceramic), Raschig Rings (Ceramic), Raschig Rings (Steel), Pall rings (Metal), Pall rings (Plastic, e.g. polypropylene) and the like. Monoliths, which are structures containing multiple, independent, vertical channels and may be constructed of various materials such as plastic, ceramic, or metals, in which the channels are typically square, are also suitable wettable structures. Other geometries could also be used.

Other materials that promote the distribution of liquid and vapors may also be used, including mist eliminators, demisters, or other wire or multi-filament type structure. Such multi-filament structures may include one or more of fiberglass, steel, Teflon, polypropylene, polyethylene, polyvinylidenedifluroride (PVDF), polyester, or other various materials, which may be knitted (or co-knit, where more than one type of filament or wire structure is used), woven, non-woven, or any other type of multi-filament structure. Structures including multifilament wires as typically used in demister services, structures including an element of woven fiberglass cloth, and high surface area stainless steel structured packings are preferred.

Reactors according to embodiments disclosed herein may include one or multiple reaction zones.

One of the primary products from processes according to embodiments herein may include dimers of the isoolefins. For example, isobutylene may be dimerized to form a C₈ tertiary olefin. In some embodiments, the dimers have 8 to 10 carbon atoms and correspond to dimers prepared from C₄ or C₅ olefins.

In prior C₄ dimerization schemes, fixed bed reactions are staged to increase the selectivity toward the C₈ dimer. The present inventors have found that through proper dimerization reaction conditions and appropriate use of a selectivator in each reactor, the need for an intermediate debutanizer may be minimal while still achieving a high isoolefin conversion, and the effluent from a first reactor may be fed directly to a second reactor, without intermediate componential separations.

Following reaction in upstream reactors, such as fixed bed reactors, the effluent from the last reactor may be fed to a catalytic distillation column reactor to separate the reaction products while targeting complete conversion of the isobutylene. Embodiments herein contemplate continued dimerization in the catalytic distillation column reactor. Other embodiments herein contemplate etherification in the catalytic distillation column reactor.

The dimerization of isoolefins may be carried out in a partial liquid phase in the presence of an acid cation resin catalyst either in straight pass type reaction or in a catalytic distillation reaction where there is both a vapor and liquid phase and a concurrent reaction/fractionation. Catalysts used in dimerization reactors may include acid resins, such as AMBERLYST 15 (available from Rohm and Haas) or related oleum derived resins and may include phosphoric acid derived catalysts, such as those known to the industry as SPA (solid phosphoric acid) catalysts.

Oxygen-containing moderators may be used to influence the selectivity of the dimerization reaction to the dimer product. Oxygen-containing moderators useful in embodiments disclosed herein may include water as well as tertiary alcohols and ethers. For example, the oxygen-containing moderator may include at least one of: water, tertiary butyl alcohol, methanol, methyl tertiary butyl ether, ethanol, and ethyl tertiary butyl ether.

Dimerization reactions carried out in the presence of the oxygen-containing moderators may concurrently produce dimers, and some trimers, of the isoolefins, and various oxygen-containing byproducts due to reaction of a moderator with an isoolefin or an isoolefin dimer. For example, the oxygenated dimerization byproducts may include C₅-C₁₆ ethers and C₅-C₁₂ alcohols. Isobutylene, for example, may react with methanol (selectivator) to form methyl tert-butyl ether (MTBE). In some embodiments, 1-butene or 2-butene present may react with a moderator to form secondary ethers, such as methyl sec-butyl ether, which may be undesireable. Such dimerization process in the presence of a moderator, may eliminate the need for an intermediate deisobuanizer.

The resulting dimers may be used, for example, as a raw material for the production of various chemicals, such as herbicides and pesticides. In other embodiments, the dimer may be fed to an alkylation system, where the dimer may dissociate into constituent olefins and react with an alkane to produce an alkylate in the gasoline-boiling range. The dimer may also be hydrogenated to form gasoline-range hydrocarbons, such as iso-octane, iso-nonane, and other hydrocarbons. In yet other embodiments, the dimer containing stream may be used as a gasoline-range hydrocarbon blendstock without hydrogenation or alkylation.

Operating conditions within catalytic distillation reactor systems for dimerizing isoolefins may include temperatures and pressures sufficient for a) recovery of the unreacted C4 and/or C5 hydrocarbons, water, and other light components as an overhead vapor fraction, b) the desired reactivity of the isoolefins over the catalyst, and c) recovery of the dimer as a bottoms liquid fraction. The temperature within the reaction zone may thus be intimately linked to the pressure, the combination of which provides for boiling of the isoolefin and water within the reaction zone(s). Higher temperatures may be required in portions of the column below the reaction zone, thus providing for the separation of the dimer from the unreacted feed compounds.

Typical conditions for the catalytic distillation MTBE reaction include catalyst bed temperatures of about 150-170° F., overhead pressures of about 80-130 psig and equivalent liquid hourly space velocities of about 1.0 to 2.0 hr⁻¹. The temperature in the column is determined by the boiling point of the liquid mixture present at any given pressure. The temperature in the lower portions of the column will reflect the constitution of the material in that portion of the column, which will be higher than the overhead; that is, at constant pressure a change in the temperature indicates a change in the composition in the column. To change the temperature, the pressure in the column may be changed. Temperature control in the reaction zone is thus controlled by the pressure with the addition of heat (the reactions being exothermic) only causing more boil up. By increasing the pressure the temperature is increased, and vice versa. Even though a distillation column reactor is used, some of the isoolefin may be unconverted and may exits the column with the overheads.

The ether product, being the highest boiling material, is removed from the distillation column reactor as a bottoms, along with the dimers in the effluent from the upstream reactors. The overheads may contain unreacted light alcohols, such as methanol or ethanol used as a selectivator in the upstream reactors and/or a reactant in the distillation column reactor, and isoolefin along with light inerts, such as normal butene and butanes or pentene and pentanes.

Referring now to the FIGURE, a simplified process flow diagram of a system for the dimerization and/or etherification of isoolefins according to embodiments disclosed herein is illustrated.

When operating in dimerization mode, a hydrocarbon feed, such as a raffinate from a butadiene separation process, comprising isoolefins, such as isobutylene, and one or more of isobutane, 1-butene, butadiene, n-butane, and 2-butene, may be fed via a flow line 101, to a reactor 10, such as a fixed bed reaction system containing a resin catalyst suitable for dimerization and etherification reactions. In some embodiments, the butadiene in the feedstock may be limited to less than 3000 ppm via an upstream process such as a hydrogenation process. A reaction moderator, such as oxygenates, may also be fed to reactor 10 via a flow line 400. Alternatively, and/or additionally, methanol may be fed to reactor 10 via flow line 304. Such methanol may come from an upstream methanol rectification section, which is not illustrated.

In reactor 10, the isobutylene reacts in the presence of the catalyst contained in the reaction zone to convert a portion of the isobutylene to dimers of isobutylene such as isooctene, and/or ethers, such as MTBE.

The effluent 105 from reactor 10 may then be combined with additional reaction moderator (e.g., oxygenates) 400 and/or methanol 304 and fed to reactor 20, also containing a resin catalyst suitable for dimerization and etherification reactions. In reactor 20, the isobutylene reacts in the presence of the catalyst contained in the reaction zone to convert a portion of the isobutylene to form additional dimers, including dimers of isobutylene such as isooctene, and/or additional ethers, such as MTBE, in addition to those produced in reactor 10. Feeding effluent 105 to reactor 20 may, in some embodiments, be done without the step of an intermediate debutanizer.

The effluent 204 from reactor 20 may then be fed to a catalytic distillation column 30. If necessary or desired, additional methanol 301 may be fed to catalytic distillation column 30. The feed of the effluent 204 from reactor 20 may be introduced to the catalytic distillation column 30 below the reaction zone containing a catalyst suitable for dimerization and/or etherification reactions. Such catalyst may be the same, or different, from the catalyst in reactors 10 and 20. The heavier reaction products may distill downward, and the isobutylene and lighter components upward into the reaction zone, where the isobutylene reacts in the presence of the catalyst contained in the reaction zone to convert a portion of the isobutylene to dimers of isobutylene such as isooctene, and/or ethers, such as MTBE.

The overhead distillate 306 from catalytic distillation reactor 30 may include unreacted C4s, such as n-butane, 2-butene, and 1-butene, as well as unreacted methanol and isobutylene, and be sent to one or more downstream processes such as methanol extraction and recovery, alkylation, isomerization, or metathesis processes.

As a side reaction in one or more of reactors 10, 20, and 30, the moderator (oxygenate) may react with a portion of at least one of the isoolefin, 2-butene, and 1-butene present in the reaction zones to form oxygenated byproducts, such as methyl sec-butyl ether (MSBE).

The catalytic distillation column bottoms 206 may include dimers, and some trimers, produced via reaction in reactors 10, 20, and 30, and may be used as a raw material for various downstream processes. For example, a resulting dimer fraction may be used as a raw material for the production of various chemicals, such as herbicides and pesticides. In other embodiments, the dimers may be fed to an alkylation system, where the dimers may dissociate into constituent olefins and react with an alkane to produce an alkylate in the gasoline-boiling range. The dimer may also be hydrogenated to form gasoline-range hydrocarbons, such as octane, nonane, and other hydrocarbons. In yet other embodiments, the dimer containing stream may be used as a gasoline-range hydrocarbon blendstock without hydrogenation or alkylation. Due to the low concentration of linear butenes in the feed to the dimerization unit, it may not be necessary to remove the oxygenated byproducts from the dimerization effluent prior to these downstream processes.

Alternatively, the catalytic distillation column bottoms 206 may be further separated, such as in columns 40 and 50. The catalytic distillation column bottoms 206, which includes dimers and trimers, MTBE and MSBE, as well as unreacted oxygenates may be sent to a first fractionation column 40. The overheard product stream 401, which may include any unreacted oxygenates as well as MTBE and MSBE, may be recycled to reactors 10 and/or 20 as the oxygenate moderator 400. A portion of the overhead product stream 401 may also be purged by flow line 402, or used as a fuel blend.

The bottoms product stream 402, from column 40, which may include the dimers and trimers of isobutylene, may be fed directly as export grade product, used as a fuel blend, fed to a downstream hydrogenation process, or further fractionated in column 50. Column 50 may separate the bottoms product 402 into an overhead dimers (isooctene) stream 501, and a C12+ fraction 504 which includes trimers of isobutylene.

While generally described above as producing a dimerization product, the system described herein may, in one or more embodiments, be operated in etherification mode to produce an MTBE product. When the system is used for production of MTBE, for example, columns 40 and 50 may be placed out of service by closing valve 60, and recovering an MTBE product stream via flow line 207. Further, when producing an MTBE product, as opposed to a dimerization product such as isooctene, the feed ratio of oxygenates/methanol to mixed C4s may be increased.

In one or more embodiments, the process may initially be producing dimers as a targeted product, separating the dimers of the isoolefins from unreacted oxygenates and unreacted C4s in the catalytic distillation reactor system 30, producing a bottoms stream comprising the dimers of the isoolefins, and an overhead stream comprising unreacted light oxygenates and C4s. During a first period of time, the system may be operated in dimerization mode where the oxygenates are fed to the first and second reactor at a concentration for the oxygenate to be effective as a selectivator, producing dimers of the isoolefins. After running in dimerization mode, the amount of oxygenates fed to the first and second reactors may be increased to a concentration for the oxygenate to be effective as a reactant, thereby producing ethers of the isoolefin. During etherification, the bottoms fraction via a flow line 206 may be taken directly as product, with valve 60 being closed. After running the process in etherification mode for an amount of time, the concentration of oxygenates may be reduced, valve 60 opened, and the system will then continue producing dimers.

For example, when operating in dimerization mode, the mixed C4s to oxygenates ratio may be from 5:1 to 2:1 with the oxygenates being primarily MTBE, and when operating in etherification mode, the mixed C4s to oxygenates ratio may be from 2:1 to 1:2 with the oxygenates being primarily methanol.

Accordingly, disclosed herein is a system which may flexibly produce a dimerization product or an etherification product without having to take reactors out of service. The switch from dimerization to etherification may only require the increase in oxygenates feed and, when additional separation of the resulting product is not desired, the closure of a mid-process valve (valve 60). Both processes may function with suitable high isobutylene conversion without the need for a debutanizer between the first and second reactors 10, 20.

While the system is illustrated as including two fixed bed reactors, more or fewer rectors may be used. In such embodiments, the feed of oxygenates and/or methanol may be staged so as to achieve the desired selectivity in the dimerization and/or etherification reactions.

Additionally, embodiments herein may provide for the flexibility in producing both ethers and/or isoolefins. For example, the system may be used for an extended run to produce MTBE, and then transitioned to being used for an extended run to produce isobutylene dimers.

As described above, embodiments herein provide for flexible dimerization and/or etherification of isoolefins. In certain embodiments of the present disclosure, isobutylene undergoes a controlled dimerization process in a series reactor configuration in the presence of oxygenates under mild conditions. Additional oxygenates reaction is completed in the subsequent catalytic distillation tower that supplements the requirement of the moderator in the reaction section. The advantages of a series reactor configuration includes higher production of C8 olefins and lower trimers and tetramers formation.

The present disclosure, among other things, provides a reduction in Capex by eliminating the need for a debutanizer (deB) between the 2 tubular or fixed bed reactors while maximizing the isobutylene concentration and conversion. The removal of isooctene and heavier molecules is not required to achieve high overall isobutylene conversion.

The present disclosure provides, among other things, more robust model with respect to MSBE handles, improved temperature and reaction rate controls, optimum control of moderators in the dimerization process, adjustments based on type of oxygenate (molar ratio of total oxygenates/IB), better prediction of oxygenate purge, and general optimization in equipment design.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed:

1. A process for the selective dimerization and etherification of isoolefins, the process comprising:
feeding a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates;
feeding the first reactor effluent directly to a second fixed bed reactor containing a second catalyst, producing a second reactor effluent comprising dimers of the isoolefin, unreacted C4s, and unreacted oxygenates;
feeding the second reactor effluent to a catalytic distillation reactor system containing a third catalyst;
concurrently in the catalyst distillation reactor system;
reacting unreacted C4s in the presence of the third catalyst to form additional dimers of the isoolefin and/or ethers, and
separating the dimers of the isoolefins from unreacted oxygenates and unreacted C4s, producing a bottoms stream comprising the dimers of the isoolefins, any produced trimers of the isoolefins, and heavy oxygenates, and an overhead stream comprising unreacted light oxygenates and C4s.

2. The process of claim 1, further comprising feeding the bottoms stream to a first fractionation column, and producing a second overhead stream comprising oxygenates and a second bottoms stream comprising dimers of the isoolefins and any trimers of the isoolefins.

3. The process of claim 1, further comprising feeding a second oxygenate stream to the second fixed bed reactor.

4. The process of claim 1, further comprising feeding methanol to the catalytic distillation reactor system.

5. The process of claim 1, further comprising recycling a portion of the second overhead stream to the first fixed bed reactor, the second fixed bed reactor, and/or the catalytic distillation reactor system.

6. The process of claim 2, further comprising feeding the second bottoms stream to a second fractionation column, producing a third overhead stream comprising dimers of the isoolefins and a third bottoms stream comprising trimers of the isoolefins.

7. The process of claim 6, further comprising hydrogenating the dimers of the isoolefin to form a hydrogenated product.

8. The process of claim 1, wherein the isoolefin is isobutane, and wherein the oxygenate stream comprises methanol.

9. The process of claim 1, wherein the oxygenate is fed to the first and second fixed bed reactors at a concentration for the oxygenate to act as a selectivator for the dimerization of the isoolefin, and wherein the oxygenate is fed to the catalytic distillation reactor system at a concentration to act as a reactant producing ethers.

10. The process of claim 1, wherein the oxygenate is fed to each of the first and second fixed bed reactors and the catalytic distillation reactor system at a concentration for the oxygenate to act as a selectivator for the dimerization of the isoolefin.

11. The process of claim 1, wherein the first, second, and third catalysts are each a catalyst capable of being catalytically active for both dimerization and etherification at respectively appropriate reaction conditions.

12. A process for the flexible production of dimers and ethers comprising:
feeding a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates;
feeding the first reactor effluent directly to a second fixed bed reactor containing a second catalyst, producing a second reactor effluent comprising dimers of the isoolefin, unreacted C4s, and unreacted oxygenates;
feeding the second reactor effluent to a catalytic distillation reactor system containing a third catalyst;
concurrently in the catalyst distillation reactor system;
reacting unreacted C4s in the presence of the third catalyst to form additional dimers of the isoolefin and/or ethers, and
separating the dimers of the isoolefins from uncreacted oxygenates and unreacted C4s, producing a bottoms stream comprising the dimers of the isoolefins, any produced trimers of the isoolefins, and heavy oxygenates, and an overhead stream comprising unreacted light oxygenates and C4s; and
for a first period of time, feeding the oxygenate to the first and second reactor at a concentration for the oxygenate to be effective as a selectivator, producing dimers of the isoolefins;
increasing the amount of oxygenates fed to the first and second reactors to a concentration for the oxygenate to be effective as a reactant, and for a second period of time producing ethers of the isoolefin.

13. The process of claim 12, further comprising decreasing the amount of oxygenates fed to the first and second reactors to a concentration for the oxygenate to be effective as a selectivator, and for a third period of time producing dimers of the isoolefins.

14. The process of claim 12, further comprising transitioning a temperature and/or pressure of the first and second reactors from a dimerization reaction condition used during the first period of time to an etherification reaction condition used during the second period of time.

15. The process of claim 12, further comprising, during the first period of time:
feeding the first bottoms fraction to a first fractionation column, and producing a second overhead stream comprising oxygenates and a second bottoms stream comprising dimers of the isoolefins and any trimers of the isoolefins; and
feeding the second bottoms stream to a second fractionation column, producing a third overhead stream comprising dimers of the isoolefins and a third bottoms stream comprising trimers of the isoolefins.

16. The process of claim 15, further comprising taking the first and second fractionation columns out of service during the second period of time.

17. A system for the flexible production of dimers and ethers comprising:
a first fixed bed reactor containing a first catalyst configured for reacting ng a mixed C4 stream, comprising isoolefins, and an oxygenate stream to a first fixed bed reactor containing a first catalyst, producing a first reactor effluent comprising dimers of the isoolefin, unreacted C4s, and unreacted oxygenates;
a second fixed bed reactor containing a second catalyst configured for reacting the first reactor effluent and producing a second reactor effluent comprising dimers of the isoolefin, unreacted C4s, and unreacted oxygenates;
a catalytic distillation reactor system containing a third catalyst;
concurrently in the catalyst distillation reactor system;
reacting unreacted C4s in the presence of the third catalyst to form additional dimers of the isoolefin and/or ethers, and
separating the dimers of the isoolefins from uncreacted oxygenates and unreacted C4s, producing a bottoms stream comprising the dimers of the isoolefins, any produced trimers of the isoolefins, and heavy oxygenates, and an overhead stream comprising unreacted light oxygenates and C4s; and
for a first period of time, feeding the oxygenate to the first and second reactor at a concentration for the oxygenate to be effective as a selectivator, producing dimers of the isoolefins;
increasing the amount of oxygenates fed to the first and second reactors to a concentration for the oxygenate to be effective as a reactant, and for a second period of time producing ethers of the isoolefin.

18. The system of claim 17, further comprising decreasing the amount of oxygenates fed to the first and second reactors to a concentration for the oxygenate to be effective as a selectivator, and for a third period of time producing dimers of the isoolefins.

19. The system of claim 17, further comprising transitioning a temperature and/or pressure of the first and second reactors from a dimerization reaction condition used during the first period of time to an etherification reaction condition used during the second period of time.

20. The system of claim 17, further comprising, during the first period of time:
a first fractionation column configured for fractionating the first bottoms fraction and producing a second overhead stream comprising oxygenates and a second bottoms stream comprising dimers of the isoolefins and any trimers of the isoolefins; and
a second fractionation column configured for fractionating the second bottoms stream and producing a third overhead stream comprising dimers of the isoolefins and a third bottoms stream comprising trimers of the isoolefins.

21. The system of claim 20, further comprising a valve located between the catalytic distillation reactor system and the first fractionation column configured for taking the first and second fractionation columns out of service during the second period of time.
